(19) United States
(12) Patent Application Publication
(10) Pub. No.: US 2011/0183405 A1
(43) Pub. Date: Jul. 28, 2011

(54) MODIFIED MULTIWELL PLATE FOR BIOCHEMICAL ANALYSES AND CELL CULTURE EXPERIMENTS.

(76) Inventors: Tilo Pompe, Dresden (DE); Kristina Lehmann, Berlin (DE); Mirko Nitschke, Dresden (DE); Carsten Werner, Dresden (DE)

(21) Appl. No.: 12/809,211

(22) PCT Filed: Dec. 16, 2008
(86) PCT No.: PCT/EP08/67647

§ 371 Date: Apr. 11, 2011

(30) Foreign Application Priority Data
Dec. 19, 2007 (DE) 10 2007 055 865.3

(51) Int. Cl. C12M 1/00

(52) U.S. Cl. 435/283.1

(57) ABSTRACT

The invention relates to a modified multi-well plate for biochemical analyses and cell culture experiments, which can be obtained through a method for functionalization. The method comprises the following process steps:
a) Treatment of the multi-well plate by means of an ammonia low-pressure plasma, so that reactive amino groups are formed on the plate surface;
b) Application of an aqueous or alcoholic solution of a maleic anhydride copolymer;
c) Drying the solution of the maleic anhydride copolymer on the surface;
d) Heat treatment for the covalent bonding of the maleic anhydride copolymer to the multi-well plate;
e) Rinsing with aqueous solution to eliminate unbound and water-soluble maleic anhydride copolymer
f) Heat treatment to reestablish the reactivity of the anhydride groups on the maleic anhydride copolymer.

The invention relates to a modified multi-well plate for biochemical analyses and cell culture experiments.

In particular 96-well plates of polystyrene are used in numerous applications as a substrate material for biochemical analyses and cell culture experiments. A large number of analysis instruments are therefore coordinated to the standardized size thereof. The surface properties of these and similar commercially available substrates are modified, for example, by low-pressure plasma methods in order to achieve better properties in the corresponding experiments. However, this means the conventional plates have very undefined physicochemical surface properties, which lead to unexplained and thus also uncontrolled interactions of biomolecules with the substrates in the subsequent biochemical and cytobiological experiments. It is known from some studies in the field of biomaterials science, such as for example described in McClary et al. Modulation fibroblast adhesion, spreading, and proliferation using self-assembled monolayer films of alkylthiolates on gold. J. Biomed. Mater. Res 2000; 50: 428-439, that the bonding chemistry and the type of interactions that act from the surface of the substrate, can affect the action of the attached biomolecules very severely. In many cases a change of the conformation, the orientation, the concentration and the mobility of the biomolecules on the surface can thereby occur, as also disclosed by Keselowsky et al. in Integrin binding specificity regulates biomaterial surface chemistry effects on cell differentiation, Proc. Natl. Acad. Sci. USA 2005; 102:5953-5957. As was further described, in particular by Green et al. in the publication Competitive protein adsorption as observed by surface plasmon resonance, Biomaterials 1999; 20:385 through 391, in the course of the experiments the composition of the absorbed biomolecular layer on the surface can also change in a different and undefined manner. This problem has hitherto been neglected in many experiments, which can lead to inaccurate results and interpretations.

As already explained, these problems and phenomena are well known and documented in tests on well characterized model surfaces. The lack of consideration of these known problems in laboratory practice with biochemical standard analyses and cell culture experiments is probably attributable to two reasons. On the one hand, many researchers are not sufficiently aware of the problems, because they do not have extensive knowledge in the field of materials science. At the same time, only a limited number of materials are available which permit different attachment mechanisms of biomolecules to multi-well plates, such as, for example, 96-well plates.

In order to couple biomolecules to the surfaces of the multi-well plates in a targeted manner, chemical modifications are necessary. However, only very few methods are hitherto known from the prior art for this purpose.

A second disadvantageous restriction of conventional cell culture research is that microstructured functionalizations in high-performance cell culture experiments for standard cell tests, in particular on PS 96-well plates, are not very common.

A number of interesting special features of cell behavior with lateral hindrance are known from numerous tests on model surfaces. A dependence on the size of the microstructures was thereby shown not only for the cell adhesion and the cytomorphology by Lehnert et al. in Cell behaviour on micropatterned substrata: limits of extracellular matrix geometry for spreading and adhesion, J. Cell. Sci. 2003; 117:41-52 or by Tan et al. in Effects of Channel Size, Cell Type and Matrix Composition on Pattern Integrity, Tissue Eng 2003; 9:255-267.

The cell function and cell differentiation could also be severely influenced by the microstructures. Known examples thereof are the switch between apoptosis, cell division and capillary-like tube formation of endothelial cells, which is described by Chen et al. in the publication Geometrical Control of Cell Life and Death, Science 1997; 276: 1425-1428.

WO 2007/078873 A1 discloses a method for providing multi-well plates for biochemical analyses and cell culture experiments. The multi-well plates are functionalized by means of a maleic acid copolymer that is applied as a solution of an anhydrous and apriotic solvent onto the multi-well plates first functionalized with amino groups and dried. The amino groups of the substrate are introduced by silanization. During the process, the multi-well plates are heat-treated to reestablish the anhydride groups on the maleic copolymer. The heat treatment takes place after a partial blocking of the reactive anhydride units and before the action of the polymer on the substrate.

DE 103 15 930 A1 describes a method for functionalizing artificial cell carriers with maleic anhydride copolymer, in which the production of amino groups on the substrate is carried out with a plasma process in anhydrous ammonia. The maleic acid copolymer is applied from an apriotic anhydrous solvent and the functionalized carrier is used without subsequent heat treatment.

The publication FTIR spectroscopic studies of interfacial reactions between amino functionalized silicon surfaces and molten maleic anhydride copolymers, Macromol. Chem. Phys. 1999, 200: 852-857 discloses an IR spectroscopic examination by Bayer et al. of the covalent bond of maleic acid copolymers to amino-functionalized substrates. The anhydride functions of the copolymer are thereby reconverted by recyclization by means of heating for several hours in a vacuum. Heating does not represent a final process step in the production of a functionalized multi-well plate.

Methods for the functionalization of substrates by means of maleic acid copolymers are also described in DE 103 21 042 A, US 2006/0257919 A1 and DE 100 48 822 A1.

DE 103 21 042 A discloses a sample container suitable for medical diagnostics with a carrier plate and reaction chamber for analyses.

From US 2006/0257919 A 1 a method is known for producing a substrate for binding molecules, wherein the substrate has a reactive surface, with which polymer coatings containing functional groups are covalently coupled. Furthermore, the substrate is used for binding different biomolecules to polymer-coated surfaces.

DE 100 48 822 A1 describes a method for immobilizing lipid layers on surfaces, in particular pulverulent solid bodies. The solid body surface is thereby modified with molecules such that a hydrophilic area is formed. In a second process step, lipid layers are deposited on the modified surface.

Most of the techniques for the preparation of microstructures used on model surfaces and known in the prior art are difficult to apply to 96-well plates or have a lack of surface modifications that are stable in the biofluid environment. As summarized, for example, in Falconnet et al., Surface engineering approaches to micropattern surfaces for cell-bases assays, Biomaterials 2006; 27:3044-3063, microstructures of this type are formed by techniques including lithography, microcontact printing, microfluidic technique, photoactivation, surface modifications using plasma or lasers as well as printing techniques such as ink jet or screen printing.

The object of the invention is to provide a multi-well plate for biochemical and cell culture analyses, in which the surface is functionalized in a manner such that biomolecules can be coupled on the surface of the multi-well plate in a targeted manner.

The object of the invention is attained with a modified multi-well plate for biochemical analyses and cell culture experiments which can be obtained through a method for functionalization which comprises the following process steps:
a) Treatment of the multi-well plate by means of an ammonia low-pressure plasma, so that reactive amino groups are formed on the plate surface;
b) Application of an aqueous or alcoholic solution of a maleic anhydride copolymer;
c) Drying the solution of the maleic anhydride copolymer on the surface;
d) Heat treatment for the covalent bonding of the maleic anhydride copolymer to the multi-well plate;
e) Rinsing with aqueous solution to eliminate unbound and water-soluble maleic anhydride copolymer; and
f) Heat treatment to reestablish the reactivity of the anhydride groups on the maleic anhydride copolymer.

The concept of the invention is that a standardized multi-well plate for biochemical analyses and cell culture experiments is changed in its surface properties such that desired biomolecules can be bonded covalently or non-covalently. The secondary interactions with biomolecules can be influenced in a targeted manner via the selection of the copolymer for the coating. The method used according to the invention contains the modification with maleic anhydride copolymers which contains an option for coupling numerous biomolecules, including peptides, proteins, polysaccharides and other bioactive molecules. In addition, with the targeted variation of the comonomer of the copolymers used, a different density of bondable groups can be achieved and the secondary interaction between the surface and biomolecules varied by means of polar and hydrophobic interactive forces.

Maleic anhydride copolymers preferably used are, for example: poly(styrene-alt-maleic anhydride) PSMA, poly(propene-alt-maleic anhydride) PPMA or poly(ethylene-alt-maleic anhydride) PEMA. It is important thereby that the reactivity of the anhydride groups according to step f) is reestablished after the rinsing step e). With the use of multi-well plates of polystyrene, the reestablishment of the reactivity of the anhydride groups is preferably carried out via a 48-hour heat treatment at a temperature of 90° C. However, if multi-well plates of polypropylene are used, the heat treatment can be carried out advantageously in only 2 hours at 120° C.

Advantageously, proteins, peptides and other bioactive molecules can be covalently bonded onto the anhydride groups of the copolymers spontaneously via free amino groups. Furthermore, other bioactive molecules, such as polysaccharides, can also be covalently bonded onto the anhydride groups via free hydroxyl-(OH) groups. In the embodiment of the invention with polystyrene plates, the bonding by means of the hydroxyl groups, such as with polysaccharides, to the anhydride groups is preferably realized by a subsequent 48-hour heat treatment at 90° C., while with the use of polypropylene plates the heat treatment preferably takes place at 120° C. and in only 2 hours.

Depending on the hydrolyzation state of the anhydride group, the functionalization of the multi-well plate on the coated surface can be carried out by non-covalent coupling of biomolecules as well as by covalent coupling. Thus bioactive molecules, such as proteins or polysaccharides, can be bonded on the hydrolyzed polymer surfaces adsorptively via the interplay of different intermolecular interactions.

In a further embodiment of the invention, a structuring of the surface functionalizations is also provided. In this embodiment of the invention lateral structures of the surface functionalizations in the micrometer range can thus be obtained on multi-well plates coated according to the invention. To this end, a thin layer of polyoxyethylene polyoxypropylene block copolymers is applied initially adsorptively from an aqueous solution. This is preferably covalently bonded to the copolymer layer locally in a cross-linking step by means of a low-pressure argon plasma. The use of a mask is provided for this purpose, which provides correspondingly prefabricated openings through which the low-pressure argon plasma can penetrate. Templates of silicon wafers are preferably used as masks. Non-bonded polyoxyethylene polyoxypropylene block copolymers are detached in a subsequent rinsing step in water. Laterally chemically heterogeneous structures can be advantageously obtained on the surface of the plate in this manner in a size range of 5 μm to 500 μM.

In the following functionalization steps, the surface regions of the maleic anhydride copolymer left in the region of the plate surface not coated with polyoxyethylene polyoxypropylene block copolymers are advantageously used for the already mentioned covalent and non-covalent coupling of proteins, peptides, polysaccharides and other bioactive molecules. This principle is of interest, for example, for the growth of cells under lateral restriction. Due to the protein-resistant properties of the polyoxyethylene polyoxypropylene block copolymer, for example, the following adsorption of cell adhesion proteins—and thus the cell growth—can take place only in the intermediate ranges with the functional maleic anhydride copolymers.

The method according to the invention is applicable not only for the widespread and very versatile 96-well polystyrene plates, but also for other configuration sizes such as 6-well, 12-well, 24-well and 48-well. A modification of 384-well plates is also possible.

Alternatively, apart from the currently widespread polystyrene plates, polypropylene plates recently coming into use can also be modified with this method, wherein polypropylene plates of the configuration sizes 96-well, 6-well, 12-well, 48-well or 384 well are used as multi-well plates.

Further details, features and advantages of the invention are shown by the following description of exemplary embodiments with reference to the associated drawings. They show:

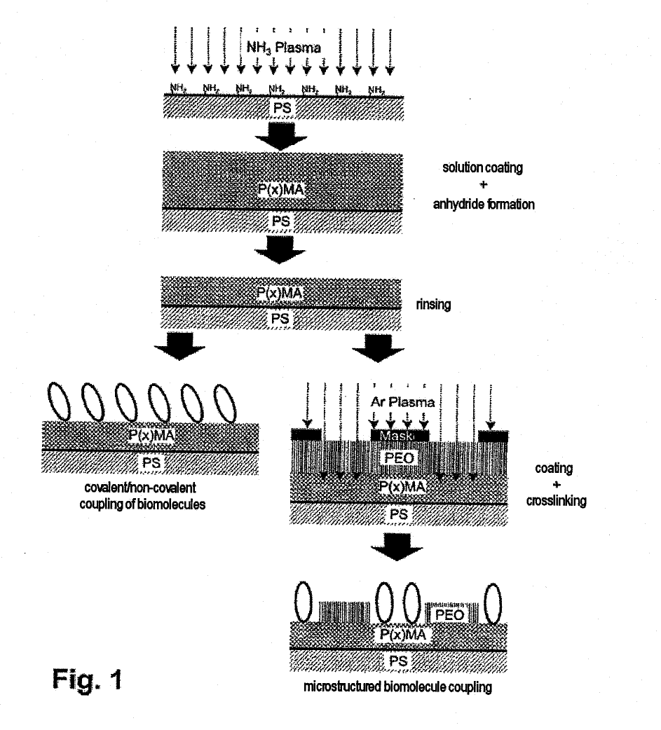

FIG. 1: A general process description for the functionalization and microstructuring of multi-well plates;

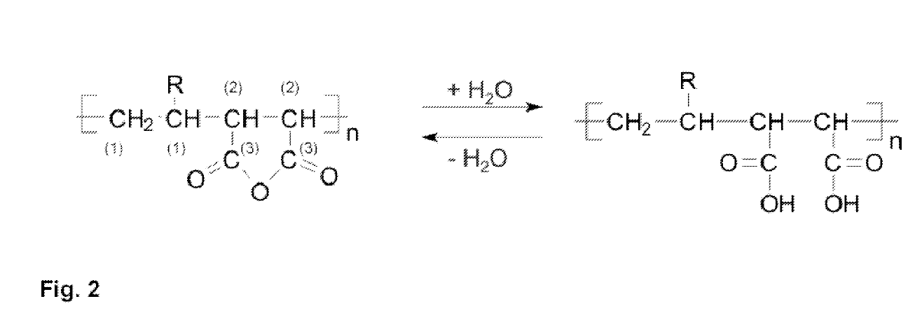

FIG. 2 An equation for the hydrolysis and the restoration of the anhydride functions to maleic anhydride copolymers;

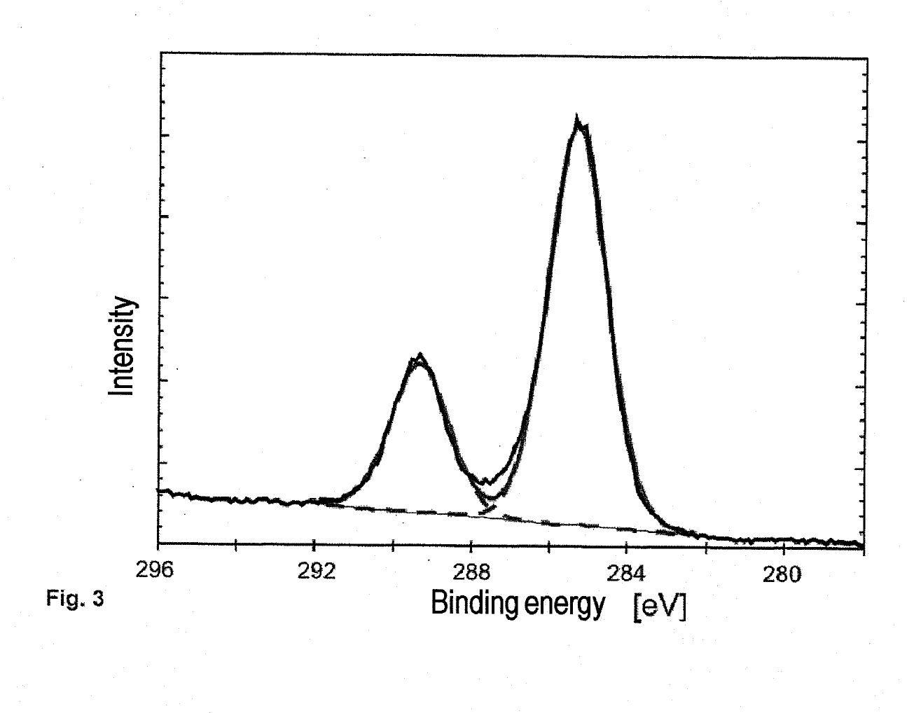

FIG. 3 A high-resolution carbon C_{1S} spectrum of a typical sample from a polystyrene (PS) surface, coated with poly(ethylene-alt-maleic anhydride) PEMA after the low-pressure ammonia plasma processing;

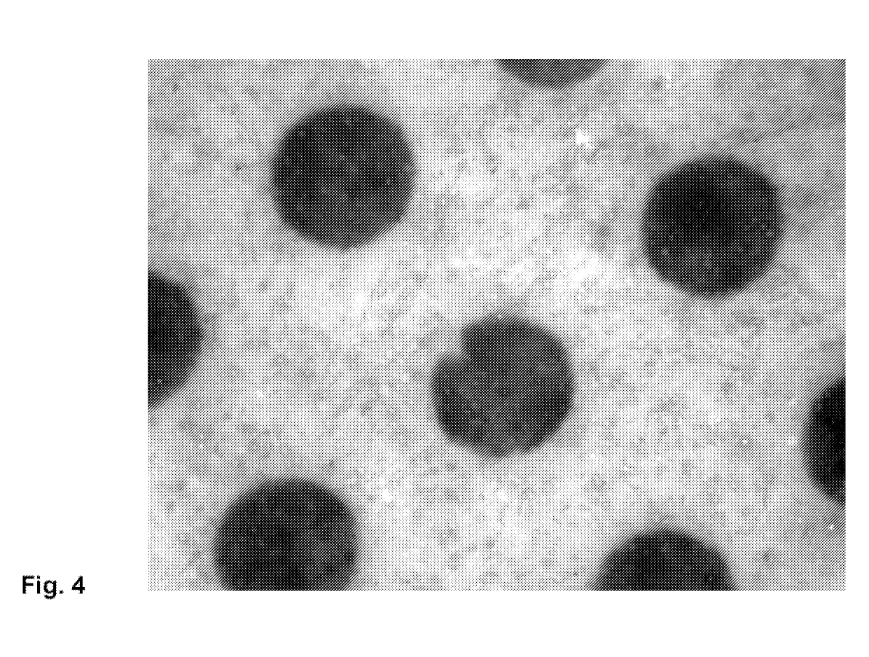

FIG. 4 A poly(ethylene-alt-maleic anhydride) functionalized polystyrene surface after a structuring with polyoxyethylene polyoxypropylene block copolymer.

Table 1: The results of the x-ray photoelectron spectroscopy (XPS) quantification of poly(ethylene-alt-maleic anhydride) PEMA, poly(propene-alt-maleic anhydride) (PPMA) on amine-functionalized polystyrene surfaces.
Table 2: The results of the detachment of thick poly(ethylene-alt-maleic anhydride) (PEMA), poly(propene-alt-maleic anhydride) (PPMA) and poly(styrene-alt-maleic anhydride) (PSMA) layers on amine-functionalized surfaces of ellipsometry measurements of the layer thickness.
Table 3: The results of the microscopic brightness measurements of fluorescent labeled bovine serum albumin after the adsorption thereof on poly(ethylene-alt-maleic anhydride) PEMA surfaces with polyoxyethylene polyoxypropylene block copolymer (PEO) structures.

According to the general process description in FIG. 1, the multi-well plate is treated by means of an ammonia low-pressure plasma such that reactive amino groups are formed on the plate surface. In the next step (solution coating and anhydride formation), an aqueous or alcoholic solution of a maleic anhydride copolymer having the general formula P(x) MA is first applied to the treated side and the solution is subsequently dried in. The covalent bond of the maleic anhydride to the multi-well plate through the regeneration of the anhydride groups is finally produced by a heat treatment.

This step is followed by rinsing with water to detach unbound and water-soluble maleic anhydride copolymer and finally the heat treatment to reestablish the reactivity of the anhydride groups on the maleic anhydride copolymer.

Depending on the hydrolyzation state of the anhydride group, the functionalization of the multi-well plate on the coated surface can be carried out by covalent or non-covalent coupling of biomolecules, as is sketched in the lower left part of FIG. 1.

On the other hand, the coating of the multi-well plate according to the lower right section of FIG. 1 can also be followed by steps for microstructuring, wherein lateral structures of the surface functionalizations in the micrometer range are obtained on the multi-well plate coated according to the invention. To this end, first polyoxyethylene polyoxypropylene block copolymer (PEO) is applied adsorptively from a solution and locally bonded in a crosslinking step by means of a low-pressure argon plasma. The use of a mask is provided for this purpose, which provides openings prefabricated accordingly, through which the low-pressure argon plasma can penetrate. In a following rinsing step unbound polyoxyethylene polyoxypropylene block copolymer is removed. Subsequently, the maleic anhydride copolymers locally left, as described, can be used for the covalent and non-covalent coupling of proteins, peptides, polysaccharides and other bioactive molecules.

As multi-well plates, polystyrene (PS) 96-well plates (μClear; Greiner Bio-One, Frickenhausen, Germany) were processed in the low-pressure ammonia plasma in order to produce free amino groups on the surface of the PS 96-well plates. Plasma processings were carried out in a computer controlled MicroSys device from Roth & Rau (Wüstenbrand, Germany). The cylindrical vacuum chamber, made of pure steel, has a diameter of 350 mm and a height of 350 mm. The low pressure, which was achieved with a turbomolecular pump, was <10⁻⁷ mbar. A 2.46 GHz electron cyclotron resonance (ECR) plasma source RR 160 from Roth & Rau with a diameter of 160 mm and a maximum output of 800 W was mounted at the tip of the chamber. The plasma source was operated in a pulsed mode. The process gases were introduced into the active volume of the plasma source through a gas flow control system. When the plasma source was switched on, the pressure was measured by a capacitive vacuometer. The samples were inserted through a load-lock system and placed on a grounded aluminum holder near the center of the chamber. The distance between the samples and the excitation volume of the plasma source was approximately 200 mm.

During the plasma treatment the power was 400 W, the pulse frequency 1000 Hz, the duty factor 5%, the anhydrous ammonia flow 15 standard cm³ per minute and the pressure 7*10⁻³ mbar. The treatment times were varied in the range from 50 s to 600 s, in order to determine optimum conditions. Based on the result of corresponding optimization tests, a time of 300 s was selected as the treatment time for the low-pressure ammonia plasma functionalization.

The hydrolyzed forms of the maleic anhydride copolymers were used for the preparation of the solutions for coating the wells. FIG. 2 thereby shows the hydrolysis and the restoration of the anhydride functions to maleic anhydride copolymers in the form of an equation for a reversible reaction. Through the hydrolysis with respectively one water molecule, a cyclic anhydride group is converted into two adjacent carboxylic groups. Accordingly, the removal of a water molecule from the adjacent carboxylic groups and a recyclization to the anhydride function occurs with the condensation. Different comonomers can be used, which differ from one another in the side chain R used. Poly(ethylene-alt-maleic anhydride) (PEMA), poly(propene-alt-maleic anhydride) (PPMA) and poly(styrene-alt-maleic anhydride) (PSMA) were used as copolymers. The side chains R correspond to hydrogen atoms (H) with PEMA and to methyl groups (—CH₃ with PPMA). In the case of PSMA, the side chains are styrene rings. PSMA has a molar mass of M_{PSMA}=20,000 g*mol⁻¹ (Leuna-Werke AG, Germany) and was dissolved in ethanol p.a. ((VWR International, Germany) to a concentration of 0.1%. PPMA with a molar mass M_{PPMA}=39,000 g*mol⁻¹ (Leuna-Werke AG, Germany) and PEMA with a molar mass of M_{PEMA}=125,000 g*mol⁻¹ (Aldrich, Munich, Germany) were dissolved in deionized water to a concentration of 0.1%. An individual well was filled respectively with 50 μl of a solution and the solution was dried therein.

After the formation of the amino groups on the polystyrene (PS) surfaces of the 96-well plates, solutions of the hydrolyzed poly(ethylene-alt-maleic anhydride) or the poly(propylene-alt-maleic anhydride) in deionized water were thus placed on the 96-well plates and dried in thereon. As already mentioned, ethanol solutions were used for hydrolyzed poly(styrene-alt-maleic anhydride) (PSMA). The maleic anhydride copolymers were hydrolyzed beforehand, wherein the anhydride in each case was converted into the carboxylic acid form in order to make it soluble in water or ethanol in each case, because nonpolar solvents, such as methyl ethyl ketone or tetrahydrofuran, would detach and damage the polystyrene (PS) surface.

The covalent bond of the copolymer to the amino groups was achieved by heating the plates for 48 hours at 90° C. A subsequent rinsing in deionized water for 24 hours was used for water-soluble copolymers (PPMA, PEMA) in order to remove unbound copolymer. Poly(styrene-alt-maleic anhydride) was carried out in aqueous phosphate buffer pH 7.4 (Sigma-Aldrich, Germany) in order to utilize the better solubility of the hydrolyzed copolymer at pH 7.4. In order to suppress a salt formation before the anhydride reconversion, these samples (PSMA) were rinsed beforehand in 0.01 n hydrochloric acid (Applichem, Germany) and subsequently in deionized water. The anhydride functions for biomolecular couplings were reactivated according to the back reaction in FIG. 2 by heating for 48 hours at 90° C.

The high-resolution carbon-C_{1S} spectrum in FIG. 3 shows by way of example for a coating with poly(ethylene-alt-maleic anhydride) PEMA, in addition to the main peak at 285.3 eV, which is to be assigned to the carbon atoms of the copolymer at positions (1) and (2) according to FIG. 2, another peak for oxygen bonded carbon in the anhydride ring at 289.4 eV, which corresponds to position (3) in FIG. 2. The ratio of both peaks [C_{289 eV}]:[C_{285 eV}] provides a further indication of the layer thickness, since over 10 nm layer thickness no carbon signals from the polystyrene substrate should usually be measured. The theoretical ratios of the two peaks [C_{289 eV}]:[C_{285 eV}] are for poly(ethylene-alt-maleic anhydride) PEMA 0.5, for poly(propene-alt-maleic anhydride) PPMA 0.4 and for a pure polystyrene (PS) surface 0. Although the theoretical value is not fully achieved, the high values in Table 1 demonstrate a layer thickness in the range of 10 nm.

In order to determine the thickness of the copolymer film, the nitrogen content and the high-resolution carbon C_{1S} spectrum, as shown in Table 1, were quantified. The nitrogen signal from the amine functionalization of the polystyrene (PS) surface can be expected as exponentially weakened by the covering with the copolymer phase, which is to be attributed to the limited mean free wavelength of the emitted photoelectrons. Table 1 shows the results of the x-ray photoelectron spectroscopy (XPS) quantification of PEMA and PPMA layers on amine-functionalized polystyrene surfaces of typical samples from four independent experiments. The nitrogen content and the ratio of the C_{1S} peaks at 289.4 eV and 285.3 eV is measured once before and after the rinsing in deionized water.

In order to remove the non-covalently bonded copolymers, the PEMA and PPMA coated surfaces were rinsed with deionized water for 24 hours. For PSMA this rinsing step was carried out in phosphate buffer pH 7.4. Due to the hydrolysis in the aqueous environment, the copolymers PEMA, PPMA and PSMA regain their solubility in aqueous environment and the unbound copolymer is detached from the surface. The XPS quantification according to Table 1 and the ellipsometric measurements of model samples according to Table 2 substantiate the successful rinsing step. The remaining copolymer layer is estimated from the XPS measurements at 8.4 nm for PEMA and 3.7 nm for PPMA. The ellipsometric measurements (7.8 nm for PEMA and 4.2 nm for PPMA) confirm these values and also show for PSMA a monomolecular copolymer layer of 4.4 nm thickness. The different layer thickness are explained by the different molecular weights of 125,000 g*mol⁻¹ (PEMA), 39,000 g*mol⁻¹ (PPMA) and 20,000 g*mol- (PSMA) and the different steric properties of the side chains, which leads to a different material bonding to the polystyrene surface during the deposit from the dissolved phase.

The lateral microstructuring of the functionalized surface was achieved by cross-linking a polyoxyethylene polyoxypropylene block copolymer by means of a low pressure argon plasma. To this end, the polyoxyethylene polyoxypropylene block copolymer (Pluronic F-68, BASF, Germany) was adsorbed from an aqueous solution for 1 hour. In this time a layer 3.8 nm thick adsorbs, which was proven ellipsometrically on model surfaces. During the low-pressure argon plasma with an output of 100 W, an argon gas flow of 40 standard cm³ per minute and a pressure of 5*10⁻³, round silicon masks with a diameter of 5.5 mm (GeSiM, Groβerkmannsdorf, Deutschland) with circular and strip-shaped areas of the size of 25 μm to 80 μm were used. The treatment time for the plasma cross-linking was varied in the range of 3 s to 10 s, in order to determine the optimum treatment time. On the basis of the result of corresponding optimization tests, a time of 7 was selected as the treatment time for the low-pressure argon plasma treatment. In the cross-linking step, the polyoxyethylene polyoxypropylene block copolymer was locally bonded by means of the low-pressure argon plasma. Unbound copolymer was subsequently eliminated by rinsing in deionized water for 24 hours. The remaining polyoxyethylene polyoxypropylene block copolymer layer has an ellipsometrically determined thickness of 1.1 nm and substantially reduces the protein adsorption, as can be seen in Table 3 based on the microscopic brightness measurement of fluorescent labeled bovine serum albumin. A deposit of protein takes place only the in the areas without a polyoxyethylene polyoxypropylene block copolymer layer, so that areas in the micrometer range are formed, which are covered with protein. If the protein fibronectin, for example, is coupled to this area instead of albumin, cells growing adherently, such as, for example, endothelial cells, can grow in these structures.

FIG. 4 shows a surface after a microstructuring with subsequent bonding of fluorescent labeled bovine serum albumin. Subsequently no protein, that is in this case fluorescent labeled bovine serum albumin, can be bonded in the locally coated areas. The coated holes at which a local protein binding is suppressed and which in FIG. 4 appear as circular areas, have a diameter of 80 μm. The areas appear dark when observed under a fluorescence microscope.

### LIST OF REFERENCE NUMBERS AND ABBREVIATIONS

1 Position of the carbon of the maleic anhydride copolymer
2 Position of the carbon of the maleic anhydride copolymer
3 Position of the carbon of the maleic anhydride copolymer
PEMA Poly(ethylene-alt-maleic anhydride)
PPMA Poly(propene-alt-maleic anhydride)
PSMA Poly(styrene-alt-maleic anhydride)
P(x)MA General abbreviation for the maleic anhydride copolymer
PEO Polyoxyethylene polyoxypropylene block copolymer

TABLE 1

| | PEMA | | PPMA | |
|---|---|---|---|---|
| | Before | After | Before | After |
| | rinsing | rinsing | rinsing | rinsing |
| N [at. %] | 0.34 | 1.10 | 0.63 | 3.6 |
| [C_{289eV}]:[C_{285 eV}] | 0.38 | 0.37 | 0.32 | 0.18 |
| Layer thickness [nm] | 13.1 | 8.4 | 10.6 | 3.7 |

TABLE 2

| Layer thickness [nm] | PEMA | PPMA | PSMA |
|---|---|---|---|
| Before rinsing | 23.3 | 17.0 | 18.9 |
| After rinsing | 7.8 | 4.2 | 4.4 |

TABLE 3

| Brightness [%] | PEMA | PEO |
|---|---|---|
| Albumin | 100 | 15% |

1. Modified multi-well plate for biochemical analyses and cell culture experiments, which can be obtained through a method for functionalization which comprises the following process steps:
a) Treatment of the multi-well plate by means of an ammonia low-pressure plasma, so that reactive amino groups are formed on the plate surface;
b) Application of an aqueous or alcoholic solution of a maleic anhydride copolymer;
c) Drying the solution of the maleic anhydride copolymer on the surface;
d) Heat treatment for the covalent bonding of the maleic anhydride copolymer to the multi-well plate;
e) Rinsing with aqueous solution to eliminate unbound and water-soluble maleic anhydride copolymer
f) Heat treatment to reestablish the reactivity of the anhydride groups on the maleic anhydride copolymer.

2. Modified multi-well plate according to claim 1, characterized in that poly(styrene-alt-maleic anhydride) PSMA, poly(propene-alt-maleic anhydride) PPMA and poly(ethylene-alt-maleic anhydride) PEMA are used as maleic anhydride copolymers.

3. Modified multi-well plate according to claim 1, characterized in that bioactive molecules are covalently bonded onto the anhydride groups.

4. Modified multi-well plate according to claim 1, characterized in that bioactive molecules are bonded adsorptively onto the polymer surfaces with hydrolyzed acid groups.

5. Modified multi-well plate according to claim 1, characterized in that peptides are covalently bonded onto the anhydride groups via free amino groups.

6. Modified multi-well plate according to claim 3, characterized in that proteins are covalently bonded onto the anhydride groups via free amino groups.

7. Modified multi-well plate according to claim 4, characterized in that proteins are bonded adsorptively onto the polymer surfaces with hydrolyzed acid groups.

8. Modified multi-well plate according to claim 3, characterized in that polysaccharides are covalently bonded onto the anhydride groups via free OH groups.

9. Modified multi-well plate according to claim 1, characterized in that the functionalization is followed by a microstructuring of the multi-well plate, in that a polyoxyethylene polyoxypropylene block copolymer layer is locally bonded in a cross-linking step by means of a low-pressure argon plasma.

10. Modified multi-well plate according to claim 9, characterized in that templates of silicon wafers are used as a mask, which have prefabricated openings through which the low-pressure argon plasma can penetrate.

11. Modified multi-well plate according to claim 9, characterized in that laterally chemically heterogeneous structures are obtained on the surfaces of the multi-well plate in a size range of 5 μm to 500 μm.

12. Modified multi-well plate according to claim 9, characterized in that bioactive molecules are covalently bonded to the anhydride groups on the surface regions of the maleic anhydride copolymer left.

13. Modified multi-well plate according to claim 12, characterized in that peptides are covalently bonded to the anhydride groups via free amino groups on the surface regions of the maleic anhydride copolymer left.

14. Modified multi-well plate according to claim 12, characterized in that proteins are covalently bonded to the anhydride groups via free amino groups on the surface regions of the maleic anhydride copolymer left.

15. Modified multi-well plate according to claim 12, characterized in that polysaccharides are covalently bonded to the anhydride groups via free OH groups on the surface regions of the maleic anhydride copolymer left.

16. Modified multi-well plate according to claim 9, characterized in that bioactive molecules are adsorptively bonded to the polymer surfaces with hydrolyzed acid groups on the surface regions of the maleic anhydride copolymer left
